(19) United States
(12) Patent Application Publication
(10) Pub. No.: US 2022/0346807 A1
(43) Pub. Date: Nov. 3, 2022

(54) UNDERCUTTING TOOL

(71) Applicant: H & B ELECTRONIC GMBH & CO. KG, Deckenpfronn (DE)

(72) Inventors: Tobias MORLOK, Mötzingen (DE); Wilfried WEBER, Schopfloch (DE); Dariusz PETRY, Aidlingen (DE)

(73) Assignee: H & B ELECTRONIC GMBH & CO. KG, Deckenpfronn (DE)

(21) Appl. No.: 17/765,967

(22) PCT Filed: Jan. 29, 2021
(86) PCT No.: PCT/EP2021/052157

§ 371 Date: Apr. 1, 2022

(30) Foreign Application Priority Data
Apr. 28, 2020 (DE) 10 2020 111 483.4

(51) Int. Cl. A61B 17/16

(52) U.S. Cl. CPC A61B 17/1617

(57) ABSTRACT

An undercutting tool for machining an undercut anchoring cut-out into a material, in particular a bone material, has a drive element, which is driveable in rotation about a main axis for application of a driving torque, and a rotatably mounted tool carrier, which has a working end with a working tool and is connectable to the drive element by means of a drive connection. The tool carrier is pivotable from an initial position, in which it extends along the main axis, into an undercutting position, in which it has an angle of inclination relative to the main axis and can be driven with a wobbling movement.

The invention relates to an undercutting tool for machining an undercut anchoring cut-out in a material according to the preamble of claim 1. In particular, the material is formed by a bone material, into which the undercut anchoring cut-out is machined in order to attach a tissue strand to it, such as a tendon or a ligament. In addition, the invention relates to a method for machining an undercut anchoring cut-out into a material using the undercutting tool. For the machining of the anchoring cut-out, the undercutting tool has a drive element, which is driveable in rotation about a main axis for the torque proof connection with a tool holder of a machine tool or for a manual drive. In addition, the undercutting tool has a rotatably mounted tool carrier, which has a working end with a working tool and is connected to the shank via a drive connection and can be subjected to a torque by the latter.

An undercutting tool is known from DE102006007232A1, with which an undercut cut-out can be made in a bone or a tooth. For this purpose, the undercutting tool has a cylindrical base body, on the front end of which, with respect to the working direction, two working projections are held, which can be pivoted away from one another by a wedge element, which can be displaced on an axial thread. To produce the cut-out, the working projections are first arranged in such a way that they form a cylindrical outer surface, with which a blind hole can be drilled in the bone. The working projections are then pivoted away from one another by displacement of the wedge element while the drill is being driven at the same time, in order to thereby produce the undercut.

A disadvantage of the known undercutting tool is that only cut-outs with a relatively large minimum diameter can be produced with this tool due to the required minimum material thicknesses for the working projections, the wedge element and the axial thread. In addition, with the known undercutting tools, a relatively large part of the bone material scraped off during the machining of the undercut remains in the drill hole. This can lead to disruptions when the working projections are moved back into the initial position, whereby in turn the removal of the undercutting tool from the drilled hole is blocked.

The object of the invention is to avoid the disadvantages mentioned in an undercutting tool of this type and also to be able to produce relatively small cut-outs with this tool.

This object is achieved by an undercutting tool having the features of claim 1. Hereby, the tool carrier is pivotable from an initial position or a drilling position, in which it extends along the main axis, into an undercutting position, in which it has an angle of inclination relative to the main axis. As a result, the tool carrier and with it the working tool can be driven in the undercutting position with a wobbling movement, in which the longitudinal axis of the working tool revolves in the manner of a precession on the surface of an imaginary cone with a fixed cone axis. By increasing the angle of inclination or the precession, the cut-out can be progressively produced starting from a blind hole with a relatively filigree working tool, which in turn allows relatively small cut-outs to be produced. In addition, such an undercutting tool allows the bone material that has been scraped off to be removed more easily and the working tool to be removed more easily from the cut-out after it has been completed.

In a particularly preferred embodiment, the tool carrier is adjustable between the initial position and the undercutting position by means of a link control. The link control allows hereby a particularly stable change in the angle of inclination of the tool carrier and the working tool provided on it. Hereby, in the undercutting position, an advantageous course of change in the angle of inclination can be predetermined via the link control. This allows, for example, that during the progressive production of the undercut cut-out, the advance speed or the contact pressure against the material to be scraped is adapted to the resistance that the respective material is expected to exert on the working tool across its different angles of inclination. In this way, a particularly precise machining of the cut-out into the respective material can be achieved.

Advantageously, the link control has a link guide, along which a control element connected to the tool carrier is displaceable, whereby the tool carrier can be deflected very directly and precisely into the respective undercutting position by means of the link guide.

In addition, it is favourable if the link guide has a first control section, which extends centrically with respect to the main axis for positioning the tool carrier in the initial position or in a drilling position for performing a drilling operation. In addition, the link guide has a second control section, which extends in an axial direction away from the main axis in an steadily increasing eccentric manner for performing an undercutting operation. As a result, both the implementation of a pure blind hole and the subsequent generation of an undercut at this blind hole can be controlled by means of a link guide.

Advantageously, the link guide also has a third control section, which extends in the axial direction towards the main axis until reaching a centrical arrangement for performing a resetting operation. In this way, the tool carrier is additionally controlled via the link control in such a way that, after the anchoring cut-out has been completed, it automatically returns to the initial position, in which it extends coaxially to the main axis and the working tool can be removed again from the cut-out easily and without jamming.

In addition, it is advantageous if the link control forms a ball-and-tube joint between the tool carrier and the drive element. The angle of inclination of the tool carrier relative to the main axis can thus be predetermined as a function of the longitudinal position of the control element within the link guide.

It is advantageous if the control element connected to the tool carrier has a spherical surface and functions as a part of the ball-and-tube joint in order to ensure stable and trouble-free deflection of the tool carrier during longitudinal displacement along the link guide.

In a particularly advantageous embodiment, the control element also has cams, which engage in longitudinal grooves in the link guide to form a torque proof connection of the drive connection. In this way, the torque can also be transmitted to the working tool via the ball-and-tube joint.

As an alternative to this, the control element is connected to a flexible drive shaft of the drive connection, for example, directly or via a drive connection, in order to allow simpler production of the control element and the link guide.

Advantageously, the link guide is also held at a housing and is displaceable along the main axis in order to ensure stable positioning of the control element within the link guide and thereby ensure precise deflection of the tool carrier.

In addition, it is favourable if the link guide is provided at a link carrier, which is accommodated in the housing and has an external thread with which a combing device of the housing can be brought into meshing engagement. In this way, the link guide can be displaced along the main axis by rotating the link carrier, to thereby deflect the control element in the desired manner.

In an advantageous embodiment, the housing is held rotatably relative to the drive element/to the drive connection. This allows the undercutting tool to be held via the housing during operation, allowing the user to guide it more stably and with a better sense of work progress.

Hereby, it is favourable, if the combing device is formed by an internal thread of the housing, which is permanently in meshing engagement with the external thread of the link carrier. As a result, the link carrier is mounted and guided at the housing in a particularly stable manner. By rotating the link carrier relative to the housing, the link guide can thus be displaced very stably along the main axis, in order to set the angle of inclination of the tool carrier or the working tool. In addition, the link carrier can also be replaced in this way in order to be able to provide different deflection characteristics of the working tool through differently shaped link guides.

As an alternative to this, the combing device has a combing element, which is adjustable between a combing position and a passive position. In the combing position, the combing element engages in the external thread and meshes with it. In the passive position, on the other hand, it is disengaged from the external thread. In this way, the displacement of the link carrier along the main axis generated during the rotary movement relative to the housing can be switched on or off as required.

It is particularly favourable hereby if an annular groove is lowered into the link carrier and the combing device has a holding element, which is adjustable between a holding position and a release position. In the holding position, the holding element is in engagement with the annular groove. In the release position, on the other hand, it is disengaged from the annular groove. In the holding position, the holding element is thus positioned in the annular groove, as a result of which the link carrier, which rotates during operation, is held in the respective longitudinal position relative to the housing. In this way, either the drilling operation or an undercutting operation can be performed at a constant height and with a constant angle of inclination.

It is advantageous if the combing device has a threaded element, which is adjustable between a blocking position, in which it blocks a longitudinal displacement of the link carrier, and a threaded position, in which it can be brought into meshing engagement with the external thread. This allows trouble-free switching to the undercutting position.

Advantageously, the combing element and/or the holding element or the threaded element is adjustable via a control handle, for example, a sleeve-shaped control handle. As a result, a particularly convenient switching of the combing element, the holding element or the threaded element is possible.

Furthermore, it is advantageous if a rotary movement of the link carrier can be blocked in the initial position in at least one direction of rotation relative to the housing, in order to avoid a screwing movement. With a corresponding application of torque, the link carrier thus remains in this end stop position or in its initial position in order to be able to introduce the working tool into a blind hole or to be able to perform the drilling operation. By applying a torque in the opposite direction of rotation, the relative rotation of the link carrier with respect to the housing or the longitudinal displacement of the link carrier along the main axis can then be generated. In this way, the displacement of the control element along the link guide and thereby the deflection of the tool carrier for performing the undercutting operation is initiated.

In this case, it is advantageous if the rotational movement of the link carrier can be blocked by means of an end stop, which is provided at the housing or is firmly connected to it. In this way, the rotationally blocking can be generated particularly easily and stably. The end stop is formed between the housing and the link carrier and blocks a relative rotational movement between the two elements in one of the two directions of rotation of the machine tool used, while the rotational movement in the other direction of rotation is free.

As an alternative to this, it is favourable if the rotational movement of the link carrier can be blocked in relation to the housing by means of a releasable locking device. As a result, the change from blocking to rotary movement of the link carrier and thus the change from drilling operation to undercut operation of the undercutting tool can be generated independently of the direction of rotation.

Advantageously, the tool carrier is also rotatably and pivotably held at the housing via a ball joint, whereby the tool carrier is mounted stably at all angles and the forces occurring at it can be safely dissipated via the housing.

It is advantageous if the tool carrier has a spherical joint section for producing the ball joint, which is formed in one piece together with the control element and the working end of the tool carrier. Due to the one-piece design, a particularly direct and stable deflection of the working end can be guaranteed via the control element controlled by means of the link guide.

Advantageously, the tool carrier formed in one piece is replaceably held at the housing in order to allow easier replacement of a worn working tool and easier cleaning or easier replacement of the undercutting tool. For better cleaning and disinfection of the working tool and the undercutting tool as a whole, the tool carrier can be removed and, if necessary, replaced. In addition, the tool carrier can be formed as a single-use product, either alone or possibly together with a corresponding joint mount to form a ball joint, which is disposed of after use and replaced with a new single-use product.

Furthermore, it is advantageous if the working tool of the tool carrier is formed by a tool insert, which is replaceable at the working end, whereby only the tool insert has to be replaced after the working tool has worn down, which allows for more convenient handling and lower wear costs.

In addition, the working tool advantageously has a drill bit, which allows safe drilling into the material in question.

Furthermore, it is advantageous if the working tool has at least one scraping edge, which also allows chip removal in the radial direction with respect to the axis of rotation and thus easier and more precise production of the undercut.

A support collar, through which the working tool protrudes from the housing and which is adapted in size to a diameter of the blind hole to be produced, is advantageously provided on a front face of the undercutting tool with respect to the working direction. As a result, the support collar can be inserted into the blind hole in order to support the undercutting tool on an edge of the blind hole during the undercutting operation, in order to be able to better absorb the transverse forces occurring during operation.

In addition, it is favourable if the drive element is formed as a shank for the torque proof connection with a tool holder of a machine tool. As a result, both the blind hole and the undercut can be mechanically produced and thus with a high torque and a high rotational speed. This allows the anchoring cut-out to be produced even in relatively hard materials or materials that are difficult to machine.

As an alternative to this, the drive element can be formed as a manually operable rotary handle. In particular when producing the undercut in a bone, the operator can be given a better feeling with regard to the amount and speed when removing the material.

In a particularly preferred embodiment of the undercutting tool, a transmission gear is provided between the mechanically or manually driven drive element and the tool carrier. As a result, when the torque is applied to the drive element, a higher number of revolutions can be achieved at the working tool. Overall, this makes it easier and faster to produce the undercut or the anchoring cut-out. Any known and suitable toothed gear can be used as the transmission gear, such as a planetary gear, whose planetary gears mesh both with a ring gear and with a sun gear.

Furthermore, the above object is achieved by a method for machining an undercut anchoring cut-out into a material using an undercutting tool of any of the embodiments described above, wherein in a first step, the undercutting tool is driven by the control element of the tool carrier, which is arranged in the first control section of the link guide and is thus arranged centrically in relation to the main axis. In a second step, a relative rotary movement of the link carrier relative to the housing is generated, by which the control element is displaced along the link guide into an eccentric position and the working end of the tool carrier is pivoted away from the main axis. In this way, the initial position is first set for easier introduction of the working tool into an existing blind hole or the drilling position for producing the blind hole by drilling. The undercut can then be produced by scraping out the blind hole from the side. Both operations are possible in a continuous workflow. As a result, the undercut anchoring cut-out can be produced particularly conveniently, precisely and quickly.

It is advantageous if the second step is initiated after a depth stop has been reached in the first step. In this way, the depth of the blind hole and the anchoring cut-out can be precisely pre-set overall.

Advantageously, in the first step, the link carrier is rotationally blocked in a first direction of rotation relative to the housing by an end stop or a releasable locking device. In the second step, this rotational blocking is released by switching to the second direction of rotation. This allows a particularly convenient and quick change between the first and the second step.

It is also favourable if, in a third step, after passing through a maximum eccentric deflection point of the link guide, the control element is again displaced along the third control section into a position centric to the main axis and the undercutting tool is then removed from the produced cut-out. In this way, canting or even damage to the machined material can be prevented when removing the working tool from the anchoring cut-outs that have been produced.

It is pointed out that all features of the object according to the invention described above can be exchanged or combined with one another, provided that an exchange or a combination thereof is not ruled out for technical reasons.

An exemplary embodiment of the invention is illustrated in the figures, wherein:

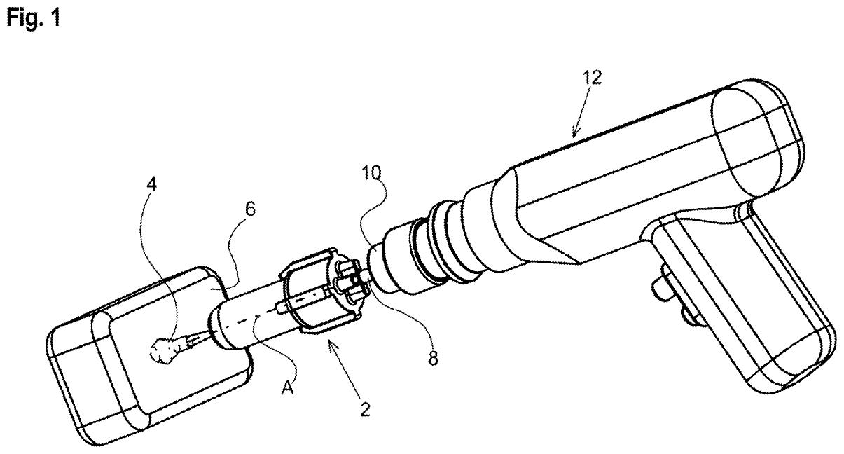

FIG. 1 shows a perspective view of an undercutting tool driven by a machine tool for producing an undercut anchoring cut-out,

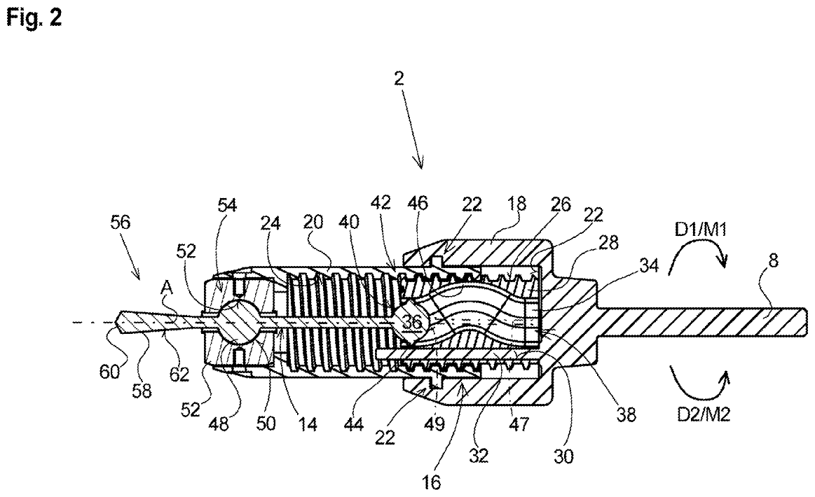

FIG. 2 shows a sectional view through the undercutting tool according to FIG. 1,

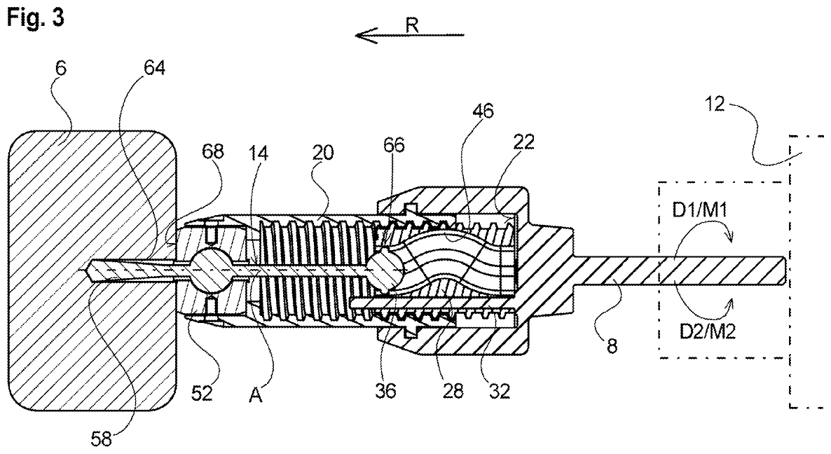

FIG. 3 shows a sectional view of the undercutting tool according to FIG. 1 at the end of a drilling operation,

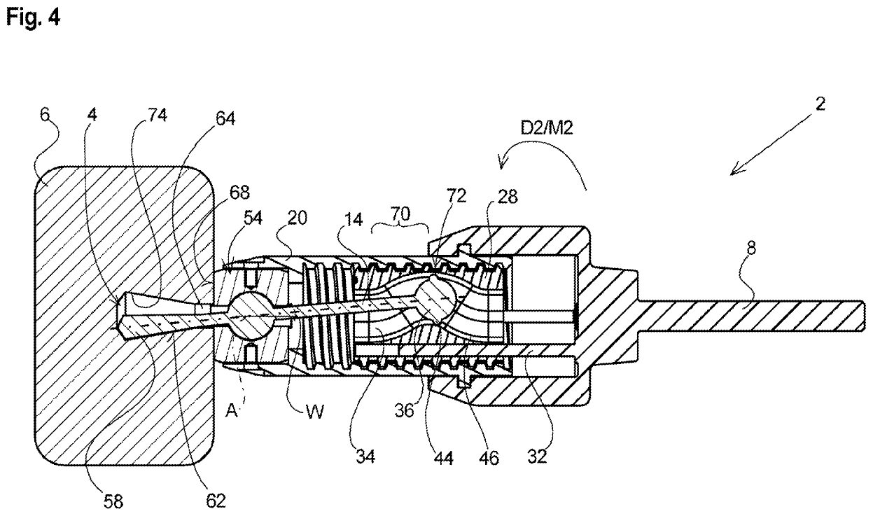

FIG. 4 shows a sectional view of the undercutting tool according to FIG. 1 during an undercutting operation,

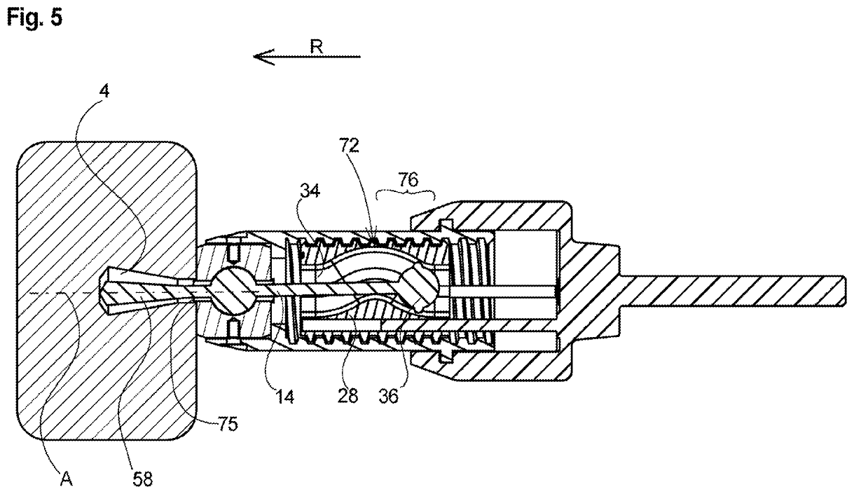

FIG. 5 shows a sectional view of the undercutting tool according to FIG. 1 in an end position,

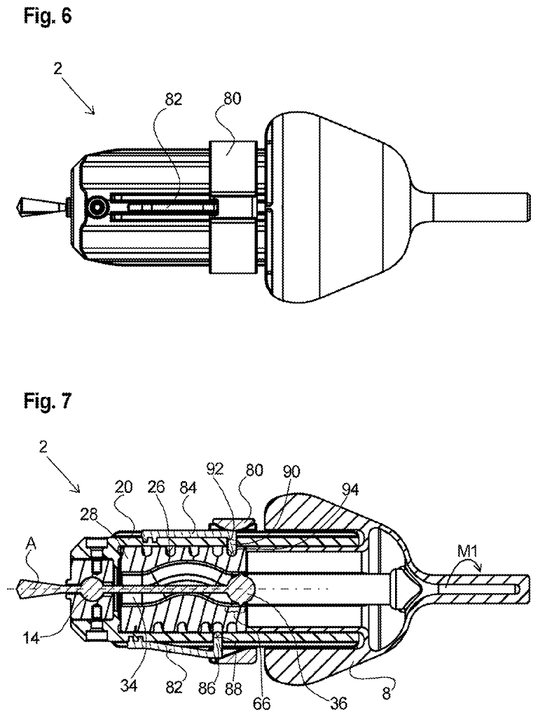

FIG. 6 shows an alternative embodiment of the undercutting tool,

FIG. 7 shows a sectional view through the undercutting tool according to FIG. 6,

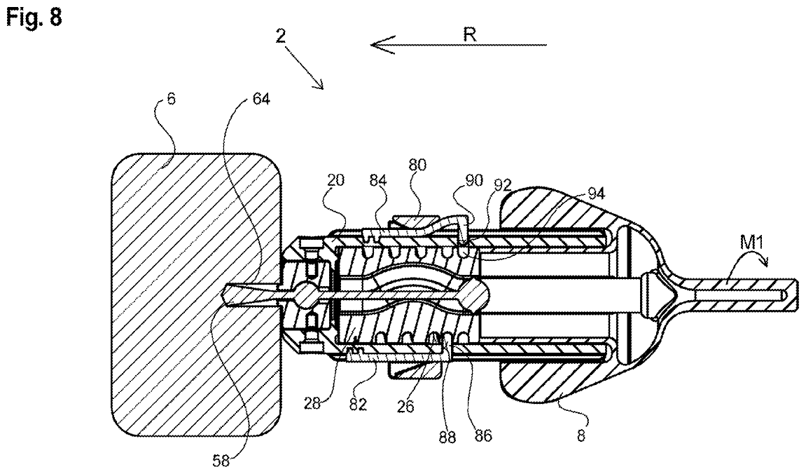

FIG. 8 shows a sectional view through the undercutting tool according to FIG. 6 in an initial position,

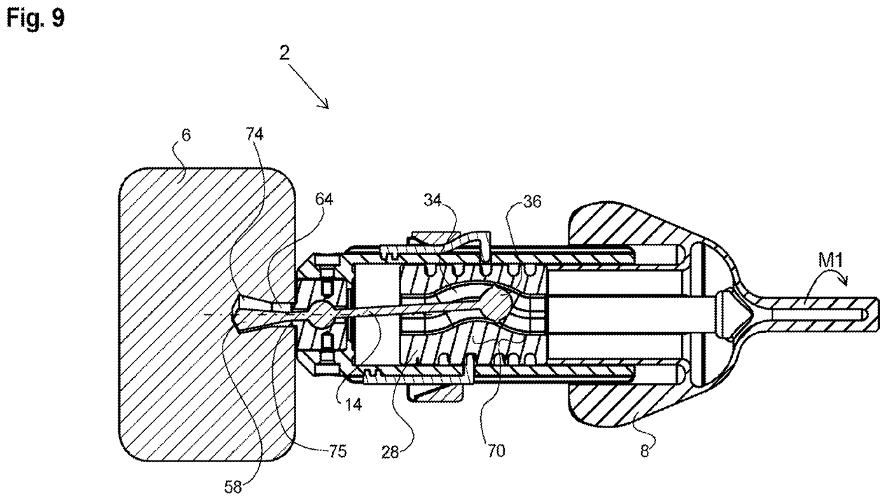

FIG. 9 shows a sectional view of the undercutting tool according to FIG. 6 during an undercutting operation,

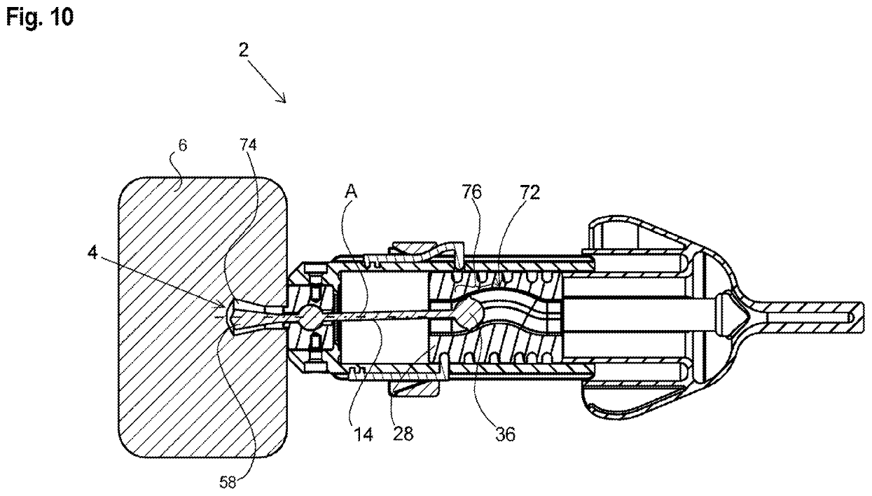

FIG. 10 shows a sectional view of the undercutting tool according to FIG. 6 before reaching an end position,

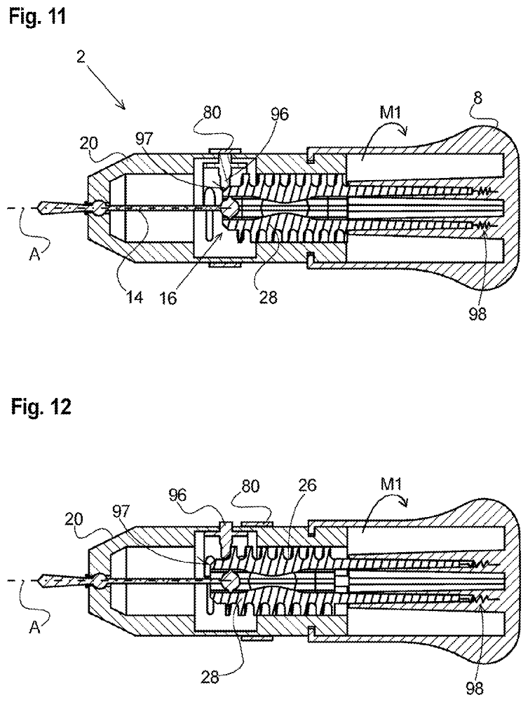

FIG. 11 shows a sectional view of the undercutting tool according to FIG. 6 with the alternative embodiment of a combing device in the drilling position,

FIG. 12 shows a sectional view of the undercutting tool according to FIG. 11 in the undercutting position,

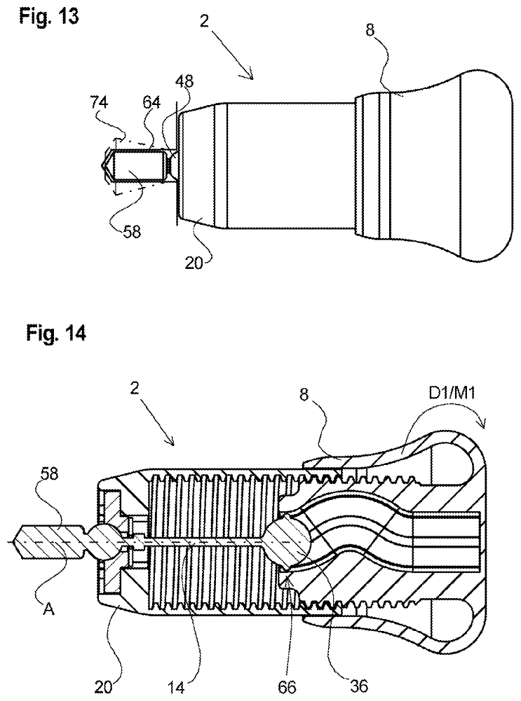

FIG. 13 shows a view of an alternative embodiment of the undercutting tool for a manual drive,

FIG. 14 shows a sectional view of the undercutting tool according to FIG. 13 in an initial position,

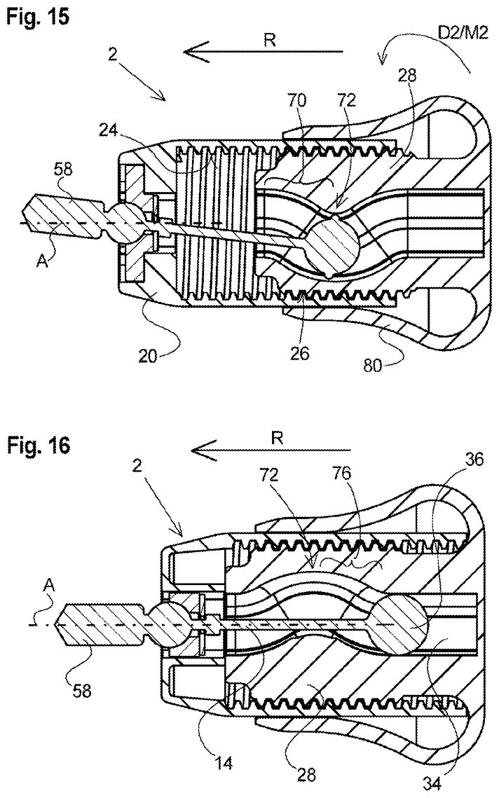

FIG. 15 shows a sectional view of the undercutting tool according to FIG. 13 in an undercutting position,

FIG. 16 shows a sectional view of the undercutting tool according to FIG. 13 in an end position,

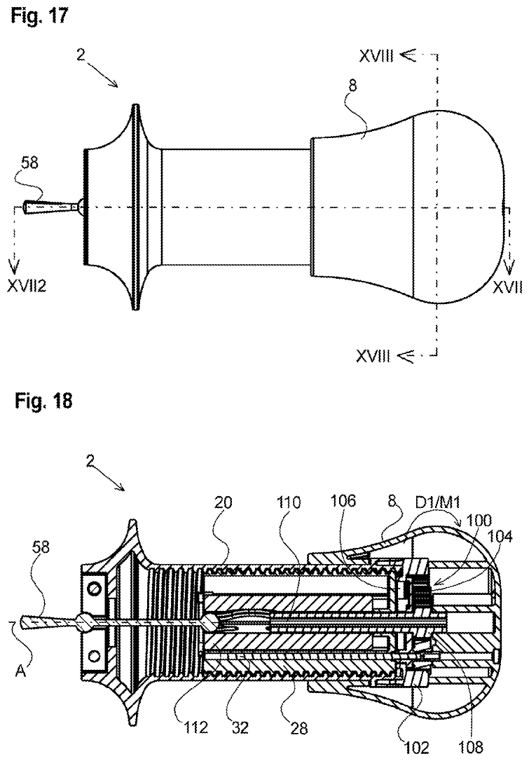

FIG. 17 shows a further alternative embodiment of the undercutting tool with a transmission gear,

FIG. 18 shows a sectional view of the undercutting tool according to FIG. 18 in plane XVII, and

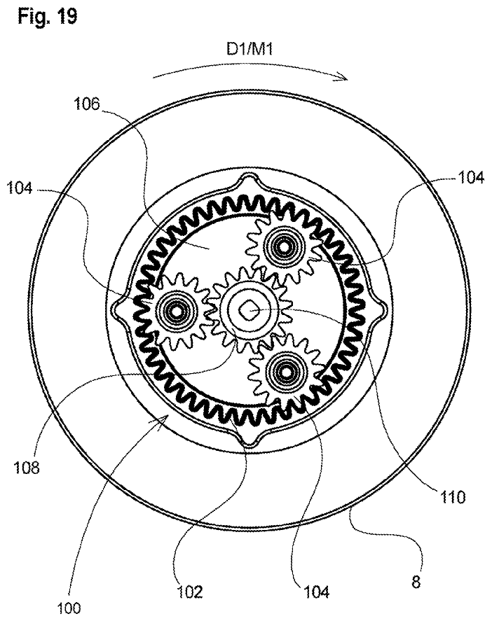

FIG. 19 shows a sectional view of the undercutting tool according to FIG. 18 in plane XVIII.

FIG. 1 shows an undercutting tool 2 for the production of an undercut anchoring cut-out 4 into a material 6. The material 6 can be formed, for example, by a bone material, wherein the anchoring cut-out is used to fix a tissue strand in a clamping matter by means of a clamping wedge (not shown). Such fastening measures are required, for example, to fix tendons or ligaments to bones, such as seen in arthroscopy or open surgery to anchor tendon transplants, ligaments or similar tissues in bone, such as in a cruciate ligament replacement.

In order to drive the undercutting tool 2 in rotation about a main axis A, it has a drive element 8 in the form of a shank, via which a torque proof connection to a tool holder 10 of a machine tool 12 can be established.

As can be seen from FIG. 2, the undercutting tool 2 has a tool carrier 14 which can be connected in a torque-proof manner to the drive element 8 via a drive connection 16. For this purpose, the drive element 8 is formed, for example, with a housing receptacle 18, in which a sleeve-shaped housing 20 is mounted.

The housing 20 has a combing device in the form of an internal thread 24, which meshes with an external thread 26 of a link carrier 28, which is formed as a screw-in sleeve and is screwed into the housing 20 up to an end stop 22 formed, for example, by the housing receptacle 18. Longitudinal guide recesses 30, in which rod-shaped catches 32 are accommodated, which extend away from the housing receptacle 18, are lowered into the link carrier 28 parallel to the main axis A.

Furthermore, a link guide 34 is lowered into the link carrier 28 along which a control element 36 is displaceable, which is formed in one piece with the tool carrier 14 and together with the link guide 34 forms a link control 38. The control element 36 has a spherical surface 40 so that it together with the link guide 34 also forms a type of ball-and-tube joint 42, which, in addition to a displacement of the control element 36 along the link guide 34, also allows the control element 36 to tilt within the same.

In addition, the control element 36 has cams 44 at the outside, which can be displaced along longitudinal grooves 46 which are lowered into the link guide 34. In this way, torques M1 and M2 can be transmitted from the link carrier 28 to the tool carrier 14 in the directions of rotation D1 and D2 about main axis A. The drive connection 16 between the drive element 8 and the tool carrier 14 is thus formed by the catch 32 connected to the housing receptacle 18, the link carrier 28 driven by this, and the longitudinal grooves 46 lowered into the link carrier 28 and interacting with the cams 44 of the control element 36.

As an alternative to this, the torques Ml, M2 can also be transmitted via a flexible drive shaft 47 as a drive connection 16. For this purpose, as shown in FIG. 2 by dash-dotted lines, the drive shaft is connected to the drive element 8 and to the control element 36, for example, directly or via an additional drive joint 49.

The control element 36 guided in the link guide 34 is connected to a joint section 48 of the tool carrier 14. The joint section 48 has a spherical surface 50 and is mounted in a spherical joint mount 52 which is provided at a front end of the housing 20 with respect to the working direction R. The spherical joint section 48 and the joint mount 52 thus form a ball joint 54, via which the tool carrier 14 can be rotated about the main axis A and also pivoted relative to it.

In addition, the tool carrier 14 protrudes with a working end 56 in the working direction R out of the housing 20. A working tool 58 is provided at the working end 56. Depending on the type of material to be machined, the working tool 58 can have a drill bit 60 and/or at least one lateral scraping edge 62.

In order to be able to replace the working tool 58 after contamination or wear and tear, it can be formed as a replaceable tool insert or as a replaceable tool bit. As an alternative to this, the working tool 58 can also be formed in one piece with the rest of the tool carrier 14, in which case the latter is then mounted on the joint mount 52 so that it can be replaced altogether, or forms together with the joint mount 52 a replaceable disposable product.

FIG. 3 shows the undercutting tool 2 at the end of a first step for producing a blind hole 64 in the material 6 by means of a drilling operation in the working direction R. The control element 36 is here arranged in a first control section 66, which extends coaxially to the main axis A. As a result, the tool carrier 14 is arranged in an initial position which corresponds to a drilling position. In this initial position, the tool carrier 14 extends coaxially along the main axis A. By applying the torque M1 to the drive element 8 by means of the machine tool 12, the control element 36 and thus the tool carrier 14 altogether is driven via the catches 32 of the link carrier 28 and via longitudinal grooves 46 in the direction of rotation Dl. As a result, the working tool 58 is driven in rotation about the main axis A and performs the drilling operation until a depth stop 68 formed by a front face of the joint mount 52 or the housing 20 is in contact with the material 6.

During the drilling operation, the link carrier 28 rests against the end stop 22 of the housing receptacle 18 and is thereby held in its longitudinal position relative to the housing 20 along the main axis A. As a result, the housing 20 is driven in rotation together with the link carrier 28 in the direction of rotation Dl.

After the depth stop 68 has been reached, the drive direction of the machine tool 12 is changed to the second direction of rotation D2 in a second step, and the link carrier 28 is thereby removed from the end stop 22. As an alternative to forming the end stop 22, the link carrier 28 can also be locked in one of the directions of rotation by a releasable locking device, which, for example, is configured as a freewheel or is manually switchable between a locking position and a release position (not shown). The switchable locking device has the advantage that both the drilling operation and the undercut generation can be done in a constant direction of rotation Dl; D2.

In any case, in this second step, by means of the catches 32 a rotational movement of link carrier 28 is generated, for example, in direction of rotation D2, while the housing 20 is held in position or at least braked, for example, by holding it manually or via the holding forces acting on depth stop 68. This generates a relative rotational movement of the link carrier 28 in relation to the housing 20, which, due to the external thread 26 meshing with the internal thread 24 of the housing 20, leads to an axial displacement of the link carrier 28 along the main axis A in the position shown in FIG. 4. As an alternative to the displacement in the working direction R shown in FIG. 4, a displacement against the working direction R can be provided with corresponding arrangement of the link carrier 28 within the housing 20 and reversed direction of rotation.

In any case, the control element 36 is displaced in a second control section 70 of the link guide 34 by the axial displacement of the link carrier 28. This extends against the working direction R and steadily increasing away from the main axis A, so that the control element 36 is deflected increasingly eccentric with increasing displacement of the link carrier 28. The tool carrier 14, which is pivotably and rotatably mounted via the ball joint 54, is thereby displaced in an undercutting position, in which it has a steadily increasing angle of incidence W relative to the main axis A until it reaches a maximum eccentric deflection point 72 shown.

During this displacement of the control element 36 beyond the second control section 70, the working tool 58 continues to be driven in the direction of rotation D2 via the catch 32, the link carrier 28 and the cams 44 interacting with its longitudinal grooves 46, which together form the drive connection 16. As a result of the drive torque M2 transmitted by the drive connection 16, the working tool 58 performs a wobbling movement, which is superimposed with a rotational movement in the manner of a precession. As a result, starting from the blind hole 64, in particular by means of the lateral scraping edge 62, an additional undercut 74 is machined into the material 6, so that the undercut anchoring cut-out 4 is formed. In order to be able to better absorb the lateral forces that occur here, a support collar 75 is provided at the depth stop 68 formed by the front face of the joint mount 52 or the housing 20, through which the working tool 58 protrudes from the housing 20 and which is adapted in size to the free diameter of the blind hole 64. As a result, the support collar can be inserted into the blind hole in order to support the undercutting tool 2 at the edge of the blind hole 64 during the undercutting operation.

After the undercut anchoring cut-out 4 has been completed and the maximum eccentric deflection point 72 has been passed through, the control element 36 is displaced in a third control section 76 of the link guide 34 by the further displacement of the link carrier 28 according to FIG. 5. This extends, for example, against the working direction R, steadily decreasing in the direction of the main axis A. As a result, the control element 36 is again arranged overall coaxially with respect to the main axis A together with the tool carrier 14 in an end position, shown in FIG. 5. In this end position, the working tool 58 can be removed from the finished anchoring cut-out 4 in the opposite direction to the working direction R without any problems and without tilting.

FIG. 6 shows an alternative embodiment of the undercutting tool 2, which works according to the same basic principle but uses a combing device, which can be adjusted for switching between the initial position and the undercutting position. For this purpose, the combing device has a control handle 80 which, according to FIG. 7, interacts with one or more combing elements 82 and one or more holding elements 84. For this purpose, the control handle 80 is formed, for example, as a sliding sleeve, which is held in a shiftable manner at the outside of the housing 20. A major advantage of this embodiment is that the housing 20 is mounted so that it can rotate freely relative to the drive element 8 and is therefore permanently held by the user during operation, and the undercutting tool 2 can be guided via the housing 20 as a result.

The at least one combing element 82 is formed as a spring pin, for example, which is also held at the outside of the housing 20. The combing element 82 has a combing section 86 at a free end, which can be placed against the external thread 26 of the link carrier 28 through a first housing opening 88 against the elastic pretension and thereby assumes a combing position. The at least one holding element 84 is also formed, for example, as a spring pin held at the outside of the housing 20. At a free end, this has a holding section 90 which, against the elastic bias, can be displaced through a second housing opening 92 into an annular groove 94, which is lowered into the link carrier 28 next to the external thread 26 and thereby assumes a holding position.

In the initial position shown in FIG. 7, the control handle 80 holds the holding section 90 of the holding element 84 in the holding position in engagement with the annular groove 94. At the same time, the control handle 80 releases the combing element 82 to the extent that the combing section 86 is disengaged from the external thread 26 and thus assumes a passive position.

When the torque M1 is applied to the drive element 8, the link carrier 28 is thus held in its longitudinal position with respect to the main axis A by the holding section 90 in the first step. In this longitudinal position, the control element 36 is arranged in the first control section 66 of the link guide 34 and the tool carrier 14 is held in its initial position or drilling position, in which it is aligned coaxially with the main axis A.

As soon as a desired drilling depth is reached during the production of the blind hole 64 according to FIG. 8, the control handle 80 is shifted into the position shown. In this position, it releases the holding element 84 to such an extent that the holding section 90 disengages from the annular groove 94 through the second housing opening 92 and arrives at a release position. At the same time, the control handle presses the combing element 82 inward in such a way that the combing section 86 comes into meshing engagement with the external thread 26 through the first housing opening 88 and thus assumes the combing position.

When the user is holding the housing 20 and the torque M1 is applied to the drive element 8 at the same time, the link carrier 28 is now displaced against the working direction R relative to the housing 20 as a result of the meshing engagement with the combing section 86. As an alternative to the displacement shown in FIG. 8 against the working direction R, a displacement in the working direction R can also be provided in the second step with a corresponding arrangement of the link carrier 28 within the housing 20 and opposite direction of rotation.

In any case, the control element 36 reaches the second control section 70 of the link guide 34 as a result of the shifting of the link carrier 28, according to FIG. 9. As a result, the tool carrier 14 is increasingly deflected and thus generates the wobbling movement at the working end, when the torque M1 is further applied to the drive element 8, whereby the undercut 74 is machined at the blind hole 64. In order to be able to better absorb the lateral forces that occur here, a support collar 75 is also provided in this embodiment at the depth stop 68 formed by the front face of the joint mount 52 or the housing 20, through which the working tool 58 protrudes from the housing 20 and which is adapted in size to the free diameter of the blind hole 64. As a result, the support collar can be inserted into the blind hole in order to support the undercutting tool 2 at the edge of the blind hole during the undercutting operation.

After the undercut anchoring cut-out 4 has been completed and the maximum eccentric deflection point 72 has been passed through, the control element 36 is displaced in a third control section 76 of the link guide 34 by the further displacement of the link carrier 28 according to FIG. 10. This extends, for example, in the working direction R, again steadily decreasing in the direction of the main axis A, so that the control element 36 is again arranged overall coaxially with respect to the main axis A in an end position together with the tool carrier 14. In this end position, the working tool 58 can be removed from the finished anchoring cut-out 4 in the opposite direction to the working direction R without any problems and without tilting.

FIGS. 11 and 12 show a further alternative embodiment of the combing device of the undercutting tool 2, in which at least one threaded element 96 is provided instead of the combing element 82 and the holding element 84. This is held at an inside of the housing 20 and is held in the initial position or drilling position, according to FIG. 11, by the control handle 80 in a position in which it rests against a front face 97 of the link carrier 28. In this way, the link carrier 28 is held in its longitudinal position relative to the main axis A. When the torque M1 is applied to the drive element 8, only the rotational movement is thereby transmitted to the tool carrier 14, which is arranged coaxially to the main axis A, via the drive connection 16.

To switch to the undercutting position, the sleeve-shaped control handle 80 is then displaced in such a way that the threaded element 96 is displaced radially outwards away from the contact with the front face 97. At the same time, the link carrier 28 is displaced by a spring device 98, which biases it in the working direction R, in the direction of the threaded element 96, so that this meshes with the external thread 26 of the link carrier 28, when the torque M1 is transmitted. This combing device allows a particularly trouble-free switching to the undercutting position.

FIGS. 13 to 16 show a further alternative embodiment of the undercutting tool 2, which works according to the same basic principle, but is not driven by a machine but by hand. In this embodiment, the undercutting tool 2 is only used to produce the undercut 74 at the blind hole 64 that has already been produced. For this purpose, the working tool 58 is inserted in the starting position into the existing blind hole 64 and held in such a way that, during the subsequent undercutting operation, the undercutting tool 2 can be supported at the front side at the edge of the blind hole 64, for example, via the spherical joint section 48 of the tool carrier 14 protruding from the housing 20. As a result, the lateral forces occurring during the undercutting operation can be better absorbed. The drive element 8 is here formed by a manually driven rotary handle, which is firmly connected to the link carrier 28 or is formed in one piece, as can be seen in particular from FIG. 14.

FIG. 14 shows the undercutting tool 2 in the initial position, with the control element 36 being arranged in the first control section 66, which extends coaxially to the main axis A. In this position, the working tool 58 is introduced into the already existing blind hole 64. Subsequent manual application of the torque M1 to the rotary handle in the direction of rotation D1 drives the link carrier 28 and, via the longitudinal grooves 46, the control element 36 and thus the tool carrier 14 as a whole.

As a result of this action on the drive element 8 formed as a rotary handle, the link carrier 28 is brought into a position displaced in the working direction R, according to FIG. 15, because of its external thread 26 which meshes with the internal thread 24 of the housing 20. Hereby, the control element 36 moves from the first control section 66 to the second control section 70, which extends steadily increasing away from the main axis A against the working direction R, so that the control element 36 is increasingly eccentric deflected with increasing displacement in the working direction R of the link carrier 28. The tool carrier 14, which is pivotably and rotatably mounted via the ball joint 54, is thereby displaced into the undercutting position, in which it has a steadily increasing angle of incidence W relative to the main axis A until a maximum eccentric deflection point 72 is reached.

In this undercutting position, as a result of the manual drive torque M2, the working tool 58 performs the wobbling movement in the manner of a precession, which is additionally superimposed with a rotational movement. As a result, starting from the blind hole 64, the additional undercut 74 is machined into the material 6, in particular by means of the lateral scraping edge 62, and the undercut anchoring cut-out 4 is produced.

After the undercut anchoring cut-out 4 has been completed and the maximum eccentric deflection point 72 has been passed through, the control element 36 is displaced by the further displacement of the link carrier 28 in the working direction R into the third control section 76 of the link guide 34 and via this into the end position according to FIG. 16. In this end position, the working tool 58 can then be removed from the finished anchoring cut-out 4 without any problems and without tilting.

FIGS. 17 to 19 show a further embodiment of the undercutting tool 2, in which the drive element 8 is rotationally coupled to the tool carrier 14 via a transmission gear 100. As a result, when the torque M1 is applied to the drive element 8, a higher number of revolutions can be achieved at the working tool 58. The alternative embodiment of the undercutting tool 2 is shown as an example for manual operation and has a rotary knob as the drive element 8. As an alternative to this, the undercutting tool 2 with the transmission gear 100 can also be formed with a shank for connection to a tool holder of a machine drive (not shown).

In any case, the transmission gear 100 has, for example, a ring gear 102 which is firmly connected to the housing 20. As can be seen in particular from FIG. 19, a plurality of planet gears 104, which are held on a planet carrier 106 functioning as an adapter, mesh with this ring gear 102. According to FIG. 18, the planet carrier 104 is connected on the one hand to the drive element 8 and also carries the catch 32, via which the link carrier 28 can be rotated relative to the housing 20. The planetary gears 104 mesh with a common sun gear 108, which is rotationally coupled via a profile pin 110 to a link part 112 but is connected in a linearly movable manner. The link part 112 can in turn be rotated but is mounted on the link carrier 28 in a fixed manner in the axial direction.

By applying the torque M1 to the drive element 8 in the direction of rotation D1, while at the same time holding the housing 20, the planetary gears 104 mounted on the planet carrier 106 also drive the sun gear 108 with translation also in the direction of rotation Dl. Hereby, the tool carrier 14 is also driven via the profile pin 110 and the link part 112 in the manner already described above via the control element 36 with the increased torque. At the same time, the link carrier 28 is rotated together with the drive element 8 by the catch 32 carried along on the planetary carrier 106. As a result, the link carrier 28 is rotated relative to the housing 20, causing it to be displaced along the main axis A in the axial direction. Hereby, the link part 112 is also displaced with the link carrier 28, as a result of which the control element 36 is in turn displaced along the link guide 34 in the manner already described above. In accordance with the other embodiments, the tool carrier 14 is initially increasingly deflected by this displacement of the control element 36 within the link guide 34 in order to generate the wobbling movement of the working tool for the production of the undercut 74 at the blind hole 64 when the torque M1 is further applied to the drive element 8. Here too, the tool carrier 14 is then moved back into the coaxial position with respect to the main axis A, in order to be able to remove the working tool 58 again from the anchoring cut-out, which has been produced, without any problems.

It is pointed out that all the elements and features described above of the various embodiments of the subject matter according to the invention can be exchanged or combined with one another, provided that an exchange or a combination thereof is not ruled out for technical reasons.

1.-34. (canceled)

35. An undercutting tool for machining an undercut anchoring cut-out into a material,
with a drive element, which is driveable in rotation about a main axis for application of a driving torque,
and a rotatably mounted tool carrier, which has a working end with a working tool and is connectable to the drive element by means of a drive connection,
wherein the tool carrier is pivotable from an initial position, in which it extends along the main axis, into an undercutting position, in which it has an angle of inclination relative to the main axis and can be driven with a wobbling movement in the manner of a precession.

36. The undercutting tool according to claim 35, wherein the tool carrier is adjustable between the initial position and the undercutting position by means of a link control.

37. The undercutting tool according to claim 36, wherein the link control has a link guide along which a control element connected to the tool carrier is displaceable in a guided manner.

38. The undercutting tool according to claim 36, wherein the link guide has a first control section, which extends centrically with respect to the main axis for setting the initial position, and a second control section, which extends away from the main axis in a steadily increasing eccentric manner for performing an undercutting operation.

39. The undercutting tool according to claim 38, wherein the link guide has a third control section, which extends towards the main axis until reaching a centrical arrangement for performing a resetting operation.

40. The undercutting tool according to claim 36, wherein the link control forms a ball-and-tube joint between the tool carrier and the drive element.

41. The undercutting tool according to claim 40, wherein the control element connected to the tool carrier has a spherical surface and functions as a part of the ball-and-tube joint.

42. The undercutting tool according to claim 40, wherein the control element has cams, which engage in longitudinal grooves of the link guide to form a torque proof connection of the drive connection.

43. The undercutting tool according to claim 40, wherein the control element is connected to a flexible drive shaft of the drive connection.

44. The undercutting tool according to claim 35, wherein the link guide is held at a housing and is displaceable along the main axis.

45. The undercutting tool according to claim 44, wherein the link guide is provided at a link carrier, which is accommodated in the housing and has an external thread with which a combing device of the housing can be brought into meshing engagement.

46. The undercutting tool according to claim 45, wherein the housing is held rotatably relative to the drive element.

47. The undercutting tool according to claim 45, wherein the combing device is formed by an internal thread of the housing, which is in meshing engagement with the external thread of the link carrier.

48. The undercutting tool according to claim 45, wherein the combing device has a combing element, which is adjustable between a combing position, in which it meshes with the external thread, and a passive position, in which it is disengaged from the external thread.

49. The undercutting tool according to claim 48, wherein an annular groove is lowered into the link carrier and the combing device has a holding element, which is adjustable between a holding position, in which it engages with the annular groove, and a release position, in which it is disengaged from the annular groove.

50. The undercutting tool according to claim 45, wherein the combing device has a threaded element, which is adjustable between a blocking position, in which it blocks a longitudinal displacement of the link carrier, and a threaded position, in which it can be brought into meshing engagement with the external thread.

51. The undercutting tool according to claim 48, wherein the combing element and/or the holding element or the threaded element is adjustable via a control handle.

52. The undercutting tool according to claim 45, wherein the link carrier is rotationally blockable in the initial position in at least one direction of rotation relative to the housing.

53. The undercutting tool according to claim 52, wherein the link carrier is rotationally blockable relative to the housing by means of an end stop.

54. The undercutting tool according to claim 52, wherein the link carrier is rotationally blockable relative to the housing by means of releasable locking device.

55. The undercutting tool according to claim 35, wherein the tool carrier is rotatably and pivotably held at the housing via a ball joint.

56. The undercutting tool according to claim 55, wherein, for production of the ball joint, the tool carrier has a spherical joint section, which is formed in one piece together with the control element and the working end of the tool carrier.

57. The undercutting tool according to claim 56, wherein the tool carrier formed in one piece is replaceably held at the housing.

58. The undercutting tool according to claim 57, wherein the working tool of the tool carrier is formed by a tool insert, which is replaceable at the working end.

59. The undercutting tool according to claim 58, wherein the working tool has a drill bit.

60. The undercutting tool according to claim 58, wherein the working tool has at least one scraping edge.

61. The undercutting tool according to claim 44, wherein a support collar is provided at a front face with respect to the working direction, through which the working tool protrudes from the housing and which is adapted in size to a diameter of the blind hole to be produced.

62. The undercutting tool according to claim 35, wherein the drive element is formed as a shank for the torque proof connection with a tool holder of a machine tool.

63. The undercutting tool according to claim 35, wherein the drive element is formed as a manually operable rotary handle.

64. The undercutting tool according to claim 62, wherein a transmission gear is provided between the drive element and the tool carrier.

65. A method for machining an undercut anchoring cut-out into a material using an undercutting tool according to claim 35, wherein the undercutting tool, in a first step, is driven by the control element of the tool carrier, which is centrically arranged in the first control section of the link guide, and, in a second step, a relative rotary movement of the link carrier relative to the housing is generated, by which the control element is displaced along the link guide into an eccentric position and the working end of the tool carrier is pivoted away from the main axis.

66. The method according to claim 65, wherein the second step takes place after a depth stop has been reached in the first step.

67. The method according to claim 65, wherein, in the first step, the link carrier is rotationally blocked in a first direction of rotation relative to the housing by an end stop or a releasable locking device and, in the second step, the rotational blocking is released by switching to the second direction of rotation.

68. The method according to claim 65, wherein, in a third step, after passing through a maximum eccentric deflection point of the link guide, the control element is again displaced along the third control section into a position centric to the main axis and the undercutting tool is then removed from the produced anchoring cut-out.
